(19) United States
(12) Patent Application Publication
(10) Pub. No.: US 2010/0055679 A1
(43) Pub. Date: Mar. 4, 2010

(54) Method for the Quantitative Analysis of the Number of Copies of a Pre-Determined Sequence in a Cell

(76) Inventors: Christoph Gauer, Munchen (DE); Wolfgang Mann, Neudrossenfeld (DE)

Correspondence Address: DILWORTH & BARRESE, LLP, 1000 WOODBURY ROAD, SUITE 405, WOODBURY, NY, 11797, US

(21) Appl. No.: 11/992,428

(22) PCT Filed: Aug. 7, 2006
(86) PCT No.: PCT/EP2006/007805

§ 371 Date: Oct. 19, 2009

(30) Foreign Application Priority Data
Sep. 23, 2005 (DE) 10 2005 045 560.3

(51) Int. Cl. C12Q 1/68

(52) U.S. Cl. 435/6

(57) ABSTRACT

The invention relates to a method for the quantitative analysis of the number of a pre-determined sequence, and optionally of sequences homologous to the pre-determined sequence, in a biological sample, whereby a defined quantity of a biological sample is subjected to at least one amplification reaction which is adapted in such a way as to amplify at least two non-homologous sequences contained in the pre-determined sequence. The number of different amplification products obtained is then determined and compared with a frequency distribution. The invention further relates to a kit for the quantitative analysis of the number of a pre-determined sequence in a biological sample, and a device which is especially suitable for carrying out the inventive method.

The present invention relates to a method for the quantitative determination of the number of a predetermined sequence and optionally of sequences homologous to the predetermined sequence in a biological sample, in particular for the determination of the absolute number of copies of alleles per cell, to a kit for the quantitative determination of the number of a predetermined sequence in a biological sample and also to an apparatus which is in particular suitable for carrying out the method of the invention.

In molecular diagnostics methods for the quantification of sequences, in particular for the quantitative determination of the number of copies of nucleic acid sequences per cell are gaining an ever more important role. Since a multitude of partly serious illnesses are caused by deviations from the normal number of copies of nucleic acid sequences in the genome, a reliable determination of the a number of copies of specific chromosomes or specific gene sections makes it possible to diagnose corresponding illnesses reliably at an early stage of the development.

Examples for partly serious anomalies which can be attributed to an increased number of copies of whole chromosomes are trisomy 18 (Edward's syndrome), trisomy 13 (Pätau syndrome) and also trisomy 21 (Down syndrome). For each of these illnesses the number of copies of the corresponding chromosomes 18, 13 and 21 is three per cell whereas healthy individuals only have two copies of the above-named chromosomes per cell. In all three cases the increase of the number of copies of the relevant chromosomes leads to most serious anomalies. Whereas carries of the trisomy 21 are severely handicapped in their development and partly have serious deformities the carriers of trisomy 18 and trisomy 13 mainly die within the first year of life.

In addition to illnesses which can be attributed to an increased number of copies of whole chromosomes there is also a multitude of illnesses which are known which relate to a changed number of copies of genes or gene sections.

The cause for the Huntington decease, a progressively developing neurodegenerative illness characterized by abnormal involuntary movements with increasing deterioration of the mental and physical capabilities has proved to be the series connection of more than 37 copies of a specific motif (CAG) with the predisposition to the development of the illness increasing with the number of repetitions of this motif in the genome. Further examples for instable tri-nucleotide sequences in humans are the Kennedy syndrome and the spinocerebral ataxy-1.

Moreover it is known that certain proto-oncogenes can multiply by gene amplification in the genome. Such amplifications are frequently recognizable in the chromosome set as so-called “double minutes” (D.M.) or as “homogenously staining regions” (HSR). As a result of the enormous increase of the gene copy number the associated protein can be produced in all cells in very large quantities which enables an enhanced activation of the cell proliferation—without a change of the individual gene as such. The mcy proto-oncogene is in particular said to be particularly frequently affected by the amplification.

As a result of the requirement for methods for the quantification of sequence copies in a biological sample a number of corresponding methods was proposed in the past.

One of the basic quantification methods which permit at least a statement concerning the presence or absence of nucleic acid sequences and, depending on how the method is carried out, also a conditional conclusion on the number of copies of the relevant nucleic acid sequences per cell, is the so-called FISH-method (fluorescence in situ hybridization). In this method the biological sample to be investigated is incubated after appropriate pre-treatment, i.e. denaturing with formamid and also prehybridization, with one or more different probes which were previously marked with respectively different fluorescent dyes under conditions which enable a hybridization of the probes with sequences homologous thereto in the biological sample. After the hybridization the samples are washed, with non-specific hybridization signals being eliminated. Finally the fluorescence signals of the preparation are evaluated with a fluorescence microscope. Each fluorescence signal that is present points to the presence of the sequence corresponding to the probe provided with the corresponding fluorescent marker. The intensity of the fluorescence can allow a conditional conclusion to be drawn on the number of the sequence copies in the biological sample. If, in contrast, no signal or only a signal lying below a defined threshold is received at the wavelength of one of the fluorescence-marked probes that is used then a conclusion can be drawn on the absence of the sequence corresponding to the corresponding probe in the biological sample. However, the absence of a corresponding fluorescence signal can also have its origin in that a mutation and/or a micro-deletion has taken place in the corresponding binding point of the sequence which is to be found and which is why the probe no longer binds to the predetermined sequence under the selected hybridization conditions. A further disadvantage of the named method lies in the fact that an undesired cross-hybridization which leads to incorrect results can never be fully precluded. Moreover, this method is comparatively expensive because, on the one hand, fluorescent dyes must necessarily be used and, on the other hand, because it requires a complicated apparatus such as fluorescence microscopes. Finally, the ability of this method produce to reliable results depends quite decisively on the quality of the probes that are used; reliable results are only obtained when the probes hybridize with an efficiency of more than 90% onto the binding positions corresponding thereto. From this it follows that an incorrect choice of the probes or also inadequate hybridization conditions can lead to a false result. A further disadvantage of this method lies in the fact that a minimum quantity of biological sample must be used in order to obtain an evaluatable fluorescence signal at all. Moreover, the sequence may not fall short of a minimum length. Furthermore, it is necessary for a valid result to analyze a multitude of cells which were susceptible to a hybridization. For this reason the FISH-analysis is not adequate for single cell diagnostics.

Another fluorescent-based method is the CGH-analysis (comparative genomic hybridization). In this method the nucleic acid of the sample to be analyzed is completely marked with a dye 1. The same quantity of nucleic acid of a reference sample is marked with a dye 2. The two reaction batches are jointly hybridized to a spread metaphase chromosome set, with the sequences contained in the two reaction batches competing for the binding sites to the spread chromosomes. Essentially a ratio of dye 1 to dye 2 such as 1:1 arises at all hybridization points. If the sample to be analyzed contains amplified regions (more than the usual copy number of the reference) then the dye 1 will predominate at this hybridization point. In the event of a deletion in the sample to be investigated one will only detect the dye 2 at this hybridization point. The reference measurement permits a relative statement concerning the frequency of sequences in the sample to be analyzed.

A special variant is the array-CGH in which hybridization is effected not on chromosomes but rather on immobilized sequences the physical address of which in the genome is known.

A further known method for the quantification of nucleic acid sequences is the real-time-PCR-method in which a PCR (polymerase chain reaction, i.e. in German “Polymerasekettenreaktion”) is carried out with fluorescence-marked primers and the increase of the fluorescence signal in dependence on the number of cycles is observed. The threshold value PCR-cycle (also threshold-cycle) is associated with the reaction time point at which the fluorescence signal is significantly distinguished from the background fluorescence and the PCR product formation runs exponentially. This correlates with the starting copy number of the DNA sequence to be augmented. In this manner DNA samples can be quantified relatively with respect to the comparison with a DNA dilution series. A disadvantage of this method however lies in the fact that the quantity of starting material cannot be reduced in size arbitrarily because with a few starting molecules, for example 10 to 100 copies as a starting material, the stochastic error as a result of the exponential amplification becomes very large which no longer permits a quantitative statement. Furthermore, this method requires complicated and expensive apparatus for the measurement of the fluorescence intensity.

A more recent method for the quantity of determination a nucleic acid sequence is a QF-PCR (quantitative fluorescence PCR) in which a plurality of PCRs are carried out in parallel in one PCR batch using different fluorescence-marked primers and the fluorescence-marked PCR products are subsequently analyzed by laser densitometry with an automatic DNA scanner. This method is also a relative quantification method since a comparison is made for two PCR products which are amplified in parallel in a PCR test. In order to make a quantitative comparison between two PCR products amplified alongside one another in a manner which can be relied on it is necessary that the two PCR part reactions take place with the same efficiency and that the fluorescence intensities of the reaction products are quantitatively analyzed at the time point of the exponential product amplification.

A method based on the QF-PCR-methodology for the determination of possible numerical aberrations of the chromosomes 21, 18, 13, X and Y in amniotic fluid samples has been described by Lucchini et al. in Wissenschaftliche Informationen (Scientific Information), September 2004. This method is based on the in-vitro-PCR amplification of repetitive and polymorphous STR (short tandem repeats) sequences with fluorescence-marked primers. After conclusion of the PCR the amplified PCR products are quantified by capillary electrophoresis. If chromosome-specific STR systems are used in this process, then conclusions can be drawn from the number of different PCR products that were obtained regarding the copy number of the corresponding chromosome. If, for example, three peaks are obtained in the reaction with a chromosome-specific STR system during capillary electrophoresis, with the peak heights amounting to 1:1:1 relative to one another, then the individuum investigated contains three different alleles of the corresponding chromosome (tri-allelic trisomy). If, in contrast, two peaks are obtained in the method, with the ratio of the peaks to one another amounting to 2:1, then the investigated individuum contains two like alleles of the chromosome per cell and also another allele of the chromosome (di-allelic trisomy). In the event that only two peaks are obtained with identical peak height, then the individuum has two alleles, so that no trisomy is present (heterocygote case). However, if only one peak is obtained, then the method does not allow any statement concerning the presence or absence of trisomy because this result is obtained both in the event of a mono-allelic trisomy and also in the event of a mono-allelic disomy. A method based on this technology for the detection of trisomy 13 is also disclosed in DE 101 02 687 A1. In order to be able to distinguish between a mono-allelic disomy and a mono-allelic trisomy it is proposed in this method to amplify three different STR-DNA ranges specific for the chromosome 13 with the PCR. However, this method also has the disadvantage that fluorescence-marked primers have to be used. Moreover, this method also requires the use of a minimum quantity of DNA because otherwise the stochastic error as a result of the exponential amplification is very large and no quantitative announcement is any longer possible. A further disadvantage of the named method lies finally in the fact that this only functions with a certain degree of reliability in a narrow PCR window, since the peak heights are only proportional to the ratio of the starting material in this window. Furthermore, this method also has the disadvantage that the absolute fluorescence intensity has to be determined.

In WO 2004/027089 a method is disclosed for the amplification of genetic information from a genetic material including a plurality of aliquots of genetic material which can be delimited relative to one another by means of PCR and for the determination of the copy number of different chromosomes per cell, with specific target sequences with a predetermined length being amplified in the PCR with fluorescence-marked primers for each chromosome to be determined. In order to obtain a pronouncement concerning the copy number of the chromosomes to be detected the fluorescence intensity of the PCR products obtained for the respective chromosomes is determined and the intensities obtained for the target sequences of each chromosome are compared with one another. When, for example, the intensity obtained with the PCR products specific for the chromosome 21 is the same as or at least approximately the same as the intensity obtained with the PCR products specific for the chromosome 1, then the pronouncement is made that the two above-named chromosomes are present in the same copy number in the biological sample. This method thus also necessarily assumes the use of fluorescence-marked primers and requires the quantitative determination of the fluorescence intensities of the individually obtained amplification products for the evaluation. This method also functions only in a narrow PCR window with a certain degree of reliability because the peak heights are only proportional to the ratio of the starting material in this window.

It is not possible with any of the above-named methods to determine the absolute copy number of a predetermined sequence present in 10 copies or less in a biological sample or, if required, to determine sequences homologous thereto, for example the absolute exact absolute copy number of alleles per cell. Apart from this, fluorescence-marked primers or probes must necessarily be used in this method, which is why expensive apparatuses are required for the evaluation.

The object of the present invention is to make available a method for the quantitative determination of a predetermined sequence in a biological sample, in particular for the quantitative determination of the number of a predetermined sequence and of sequences homologous thereto, for example the number of alleles in a cell, which is simple and cost-favourable to carry out and which also delivers reliable results, even with a small number of sequences to be determined, for example 10 or less, present in the biological sample to be investigated.

In accordance with the invention this object is satisfied by a method for the quantitative determination of the number of a predetermined sequence and optionally of sequences homologous to the predetermined sequence in a biological sample, in particular for the determination of the absolute number of copies of alleles per cell, including the following steps:
a) making available a defined quantity of a biological sample,
b) carrying out at least one amplification reaction, with the at least one amplification reaction being adapted to amplify at least two sequences which are not homologous to one another and which are included in the predetermined sequence,
c) determination of the number of the different amplification products that are obtained and also
d) comparison of the number of different amplification products obtained with at least one frequency distribution which was or is obtained by separate and in each case multiple carrying out of the same at least one amplification reaction and under the same reaction conditions as used in step b), with the same quantity of starting material having been used or being used in the amplification reaction as in step a) with at least two different reference samples, with the at least two different reference samples respectively having known copy number of the predetermined sequences which are different from one another as well as subsequent determination of the number of different amplification products received per reference sample.

Under homologous sequences in the sense of the present invention sequences are to be understood which can be amplified under the same amplification conditions with a primer pair from a sample, whereas non homologous sequences are those which cannot be amplified with one primer pair from a sample.

In distinction to a method in accordance with the prior art and the method of the invention it is not the absolute fluorescence intensity of the PCR products that is determined, such as for example in quantitative PCR, QF-PCR, FISH and CGH, and also compared with the fluorescence intensity of a control or reference sample in the case of FISH and CGH, but rather only the number of different PCR products that are obtained is determined and this number is compared with a frequency distribution. In this respect no fluorescence-marked primers have to be used in the method of the invention. Insofar as these are nevertheless used for the detection of the number of different PCR products that are obtained, it is not necessary to determine the fluorescence intensity of the obtained fluorescence-marked PCR products in a complicated and expensive manner, but rather it is only necessary to evaluate whether or not a fluorescence lying above a defined threshold value is present at a wavelength corresponding to the fluorescent dyes used. Accordingly, the method of the invention can be carried out simply and at favourable cost without costly apparatus for the quantitative detection of fluorescence.

The principle of the method of the invention relates to the comparison of the number of different amplification products obtained in the at least one amplification reaction with a frequency distribution obtained with respect to at least two reference samples with a known copy number of the predetermined sequence different from one another, with the at least two reference samples having been subjected, for the recording of the frequency distribution, separately from each other, to an amplification reaction in the same quantity made available as in step a) of the method of the invention, under exactly the same conditions as in step b) of the method of the invention and the number of the different amplification products that are obtained with each amplification reaction having been determined. In accordance with the invention a frequency distribution is used for the recording of which the amplification reaction for each of the at least two reference samples is carried out a plurality of times, for example ten times or hundred times. Since starting material with a known copy number of the predetermined sequence is used in the amplification reactions for the recording of the frequency distribution, conclusions can be reliably drawn from this comparison regarding the number of copies of the predetermined sequence in the biological sample to be investigated.

A further advantage of the method of the invention lies in the fact that it is largely independent of the amplification conditions and the quantity of the starting material of the biological sample. Even if—for example in the case of a PCR as the amplification reaction—only a fraction of the theoretically obtainable PCR products is obtained with the at least one amplification reaction, as a result of inadequate amplification conditions, such as for example a cycle number which is too low in the PCR or when using primers which only adequately bind to the primer binding sites, then this does not falsify the copy number result that is obtained because the same parameters where also used for the recording of the at least one frequency distribution. Accordingly, in the method of the invention, no stochastic error which falsifies the quantitative result can occur as a result of the exponential amplification when carrying out the method of the invention, even when using the smallest DNA starting quantities, since any such effects are leveled out by the frequency distribution. Accordingly, the method of the invention is also suitable for very small quantities of starting material.

The method of the invention is basically suitable for the quantitative determination of the number of a predetermined sequence in a biological sample, independent of the type of the predetermined sequence. The predetermined sequence is preferably a nucleic acid sequence, nevertheless it is basically also conceivable that the method of the invention can be used to detect different sequence variants of proteins or peptides. Particularly good results are obtained when the predetermined sequence is a chromosome, a gene or a gene section.

The method of the invention is also not limited with respect to the type of the at least one amplification reaction, on the contrary all conceivable amplification reactions can be used with which the existence of sequence variants can be shown. Nevertheless, it has proved advantageous to carry out a PCR as at least one amplification reaction, because a PCR can be carried out simply and comparatively quickly and with a low technical cost and complexity and any desired nucleic acid sequences from the biological sample can be amplified by the choice of suitable primer pairs.

In accordance with a preferred embodiment of the present invention a quantity of biological starting material is used in the at least amplification reaction in accordance with step b), which is adapted to amplify at least two sequences which are not homologous to one another which are included by the predetermined sequence and which is so small that it leads to an “allelic dropout” when carrying a PCR. Under an “allelic dropout” the person skilled in the art understands the loss of an allelic DNA fragment after a PCR amplification, caused by quantities of DNA starting material which are too small. In a heterogeneous DNA mixture, such as a sample of chromosomal DNA, specific alleles are represented with different frequency. Since the PCR amplifies exponentially, this unbalanced distribution can be so greatly enhanced that the lower concentrated allele is represented in such a small quantity in relation to the higher concentrated allele that it can no longer be detected. In order to avoid an “allelic dropout” a certain starting quantity of DNA material lying in the nano-gram range is always used, for example in forensic investigations, in order to obtain reliable results at all. In distinction to this, in this embodiment of the method of the invention it is indeed advantageous to operate below such a minimum quantity of a starting material as will be explained in more detail in the following.

Particularly good results are obtained in this embodiment when a biological sample is used in the at least one amplification reaction which contains less than 100 pg DNA, for example chromosomal DNA. Less than 50 pg DNA are in particular preferably used as starting material in the at least one amplification reaction, especially preferred less than 10 pg DNA and most particularly preferably less than 5 pg DNA, with it fundamentally being the case that the fewer base pairs contained by the nucleic acid in the biological sample, the less DNA can and should be used. Converted into cells, the above-named DNA quantities correspond to the use of less than 100 cells, preferably to less than 10 cells and in particular preferably to less than 5 cells in the at least one amplification reaction. Good results are in particular obtained when using individual cells as a biological starting material.

In distinction to the method used for example in forensic science, the method of the invention is based on a statistical approach in which it is not desired at all that each of the at least two sequences which are not homologous to one another is actually amplified in the at least one amplification reaction. Further, a situation should be achieved in which only a specific percentage of the at least two sequences which are not homologous to one another is actually amplified by the setting of the parameters of the amplification reaction, namely the use of a very small DNA quantity as a starting material and also if required a correspondingly small cycle number and/or very strict hybridization conditions are new for the primers to the primer binding sites. A frequency distribution is obtained in that the frequency distribution is carried out for at least two reference samples with a known copy number by multiply carrying out an amplification reaction under the same amplification conditions and in that one can draw reliable conclusions from the frequency distribution regarding the copy number of the predetermined sequence in the biological sample to be investigated. This statistical approach will be explained in more detail in the example of a PCR.

In the method of the invention it should for example be determined whether a specific chromosome, for example chromosome 21, is present in a cell in a copy number of 0, 1 or larger than or equal to 2. For this purpose a frequency distribution is recorded using three difference reference samples, a cell being used as a first reference sample which contains no copy of the chromosome 21, whereas a cell is used as the second reference sample which contains one copy of the chromosome 21 and a cell is used as the third reference sample which contains two identical copies of the chromosome 21. Each of the reference samples is subjected in each case with the same primer pairs and under the same PCR conditions to a PCR, with eight different primer pairs being used in each of the PCRs, with the eight primer pairs being adapted to respectively amplify a different specific sequence from the chromosome 21. All PCRs are carried out in each case under precisely the same conditions, and in each case 100 experiments are carried out for each of the three reference samples. After each PCR, the number of the different PCR products that are obtained is determined, for example the following frequency distribution being obtained:

TABLE 1
(Example for a frequency distribution
in accordance with a first embodiment)

| | Number of the different PCR | | | | | | | | |
|---|---|---|---|---|---|---|---|---|---|
| Copy number of the | products that are obtained | | | | | | | | |
| reference sample | 0 | 1 | 2 | 3 | 4 | 5 | 6 | 7 | 8 |
| n = 0 | 98 | 2 | 0 | 0 | 0 | 0 | 0 | 0 | 0 |
| n = 1 | 2 | 13 | 24 | 57 | 3 | 1 | 0 | 0 | 0 |
| n = 2 | 0 | 0 | 0 | 0 | 3 | 40 | 35 | 20 | 2 |

n = number of copies of the predetermined sequence in the reference sample

Since in each case eight different primer pairs were used in the individual PCRs which were adapted to amplify eight different specific sequences from the chromosome 21, the theoretical possible maximum number of obtained different PCR products for the case n=0, i.e. a cell without chromosome 21, is 0, for the case n=1, i.e. a cell with one chromosome 21, is 8 and for the case n=2, i.e. a cell with two like copies (homozygote case), of the chromosome 21, is likewise 8. In the heterocygote case the theoretical possible maximum number of amplification products in a diploid cell is 16, so that a considerable gain in information is obtained with the system. Since however only one cell was used during the recording of the frequency distribution record in each PCR, i.e. a quantity which leads to an “allelic dropout” when carrying out a PCR, some of the alleles contained in the biological reference samples are not amplified. Moreover, through the choice of the primer sequences and also the cycle number, the efficiency of each individual PCR was set to a value below the theoretically attainable limit of 1. For these reasons the theoretically maximum possible number of different amplification products in the frequency distribution reproduced in Table 1 is not obtained, neither with the sample with one copy of the chromosome 21 (n=1) nor in the sample with two copies of the chromosome 21 (n=2). Rather, for the case n=1, i.e. a reference sample with a copy number of 1, no amplification product was obtained in 2 from one hundred PCRs that were carried out, only 1 PCR product was obtained in 13 of 100 PCRs, 2 different PCR products were obtained in 24 of 100 PCRs, 3 different PCR products in 57 from 100 PCRs, 4 different PCR products were obtained in 3 from 100 PCRs and 5 different PCR products were obtained in 1 from 100 PCRs instead of the theoretical maximum of 8 different PCR products. For this case (n=1) there is thus a frequency distribution curve similar to a Gauss distribution with a maximum value of 3 different PCR products. A similar frequency distribution curve is also obtained in the case that reference samples are used with two copies of the chromosome (n=2) with the maximum of the frequency distribution curve however being shifted to higher values, namely from three different amplificates in the case n=1, to 5 to 6 different PCR products for the case n=2. For the third case in which a reference sample is used with 0 copies of the chromosome 21 no amplificate was found in 98% of the cases and an amplificate was obtained in only 2% of the cases. Since, in the last named case, no chromosome 21 was present in the samples, it must be an artefact that was found in this 2% in which a PCR product was obtained.

Following the recording of the frequency distribution, the method of the invention can now be carried out with a biological sample with an unknown copy number of the chromosome 21. For this purpose a cell is first made available and is subsequently used in a PCR which is carried out under precisely the same conditions as the PCRs during the recording of the frequency distribution, before the number of the different PCR products obtained in the PCR with the biological sample is determined, for example using capillary electrophoresis. Subsequently, the number of different PCR products that was obtained is compared with the frequency distribution reproduced in Table 1.

If no PCR product is obtained in the PCR with the cell to be investigated, then it can be found from the frequency distribution in accordance with table 1 that the copy number in the sample lies at 0 with a probability of more than 95%. Insofar as two or three different PCR products are obtained in the PCR, then the copy number of the chromosome 21 in the sample to be investigated lies with the required certainty at 1, whereas the copy number amounts to two with the required certainty in the case that five or more PCR products are obtained. Only in the event that one or four different PCR products are obtained in the PCR is it not possible in this specific conceptual example to decide with the required confidence how many copies of chromosome 21 are contained in the sample, but rather only the statement can be made that in the case of one PCR product either 0 or one copy of the chromosome 21 and in the case of 4 different PCR products one or two copies of the chromosome 21 are present. If one also wants to decide these cases with the required certainty, then one can separate the individual frequency distribution curves from one another by changing the PCR protocol or by an increase of the number of different PCRs.

In the above-named embodiment the frequency distribution consists of frequency distribution curves obtained with three reference samples with a defined copy number of the predetermined sequence, in this case chromosome 21, different from one another, with each of the three frequency distribution curves reproducing the probability for receiving each number of different PCR products for a defined copy number lying between 0 and the theoretically possible maximum number. As the person skilled in the art recognizes, the frequency distribution can also include only two frequency distribution curves which were obtained for example with reference samples with 0 and 1 copy of the predetermined sequence or also with 4 frequency distribution curves or more. Particularly good results are obtained when, for the determination of the frequency distribution, 4 to 20 and particularly preferably 4 to 10 reference samples are used with known copy numbers of the predetermined sequence which respectively differ from one another.

As an alternative to the above-named embodiment, the frequency distribution can also consist of the recitation of the average values of the number of different PCR products obtained with the individual reference samples during multiple determination. For the above-named case indicated with reference to Table 1 this would be:

TABLE 2
(Example for the frequency distribution
in accordance with a second embodiment)

| Copy number of the | Average value of the number of |
|---|---|
| reference sample | obtained different PCR products |
| N = 0 | 0.02 |
| N = 1 | 2.49 |
| N = 2 | 5.78 |

n = number of copies of the predetermined sequence in the reference sample

In the frequency distribution in accordance with this embodiment the standard deviation around the average value is preferably also given.

Since the method of the invention is a statistical method in which it is desired that not all the theoretically possible different amplification products are actually obtained and that the evaluation takes place by comparison of the result obtained for the biological sample to be investigated with a statistical frequency distribution, the at least one amplification reaction per reference sample is preferably carried out 2 to 1000 times, particularly preferably 10 to 250 times and especially preferred 50 to 150 times and most preferably preferred about 100 times to record the at least one frequency distribution in accordance with step d) of the method of the invention. The higher the number of amplification reactions carried out per reference sample of the frequency distribution, the higher is the statistical reliability but also however the higher is the experimental effort. Thus, for the generation of the frequency distribution used in step d) the at least one amplification reaction per reference sample, which has a known number of the predetermined sequence is preferably carried out 50 to 150 times since this ensures a high statistical reliability and on the other hand the experimental effort is comparatively small.

The determination of the frequency distribution can either take place prior to carrying out the method steps a) to c) and also in parallel thereto.

As explained, the method in accordance with the invention is not only suitable for determining the number of a predetermined sequence, for example of a special gene or chromosome in a biological sample, but rather in particular also for the determination of the number of a predetermined sequence and also of sequences homologous thereto per cell, with the homologous sequences preferably being alleles. In the last named embodiment of the present invention it is necessary to amplify at least one allele specific sequence in the at least amplification reaction in accordance with step b), by which a sequence is understood which is admittedly similar to a high degree between two alleles or homologous, but not identical. Since the number of the different PCR products obtained in step c) is determined and the number of the different alleles thereby influences the result, the copy number of the individual alleles can be determined by a comparison with the frequency distribution. Thus, the at least amplification reaction in step b) is preferably designed in such a way that the at least two sequences from the non-coded DNA range non-homologous to one another are amplified. In known manner the non-coded DNA range is substantially more polymorphous than the coded range, so that the probability of amplifying allele specific sequences there is large.

In a further embodiment of the concept of the invention it is proposed to adapt the at least one amplification reaction so that at least two highly polymorphous sequences which are not homologous to one another are amplified.

Good results are obtained in particular in cases in which the at least one amplification reaction is adapted to amplify at least two sequences which are not homologous to one another which are selected from the group consisting of STR sequences, VNTR sequences, SNP sequences and any arbitrary combinations hereof. STR or short tandem repeat sequences are highly polymorphous sequences which consist solely of 2 to 4 by long repetition units and which have a high variability between the single individuals. In distinction to this, VNTR or variable number of tandem repeat sequences consists of repeating DNA sections of approximately 15 to 30 by length, the total length of which are determined by the member of the repetitions of the basic unit. The VNTR sequences are also as a rule highly polymorphous, i.e. the number of the respective repetition units is very strongly distinguished between the different individuals. SNPs (single nucleotide polymorphism) are the simplest polymorphisms in which the homologous sequences are only distinguished by a base. These are also excellently suited for the carrying out of the method of the invention. Apart from this, however all other highly polymorphous sequences are suitable as markers for the method of the invention.

Furthermore, it is preferred that the at least one amplification reaction is adapted to amplify at least two sequences which are not homologous to one another which respectively only arise once per allele in the genome of the donor.

Through the number of the at least two sequences which are not homologous to one another, the position and to a certain extent also the width of the individual frequency distribution curves of the frequency distribution can be set. The at least one amplification reaction is preferably adapted to amplify between 2 and 200 sequences which are not homologous to one another, with particularly good results being in particular obtained on adaptation of the amplification of 2 to 20 sequences which are not homologous to one another, particularly preferably 3 to 15 sequences which are not homologous to one another and quite especially preferred between 5 to 12 sequences which are not homologous to one another. If the number of the non-homologous sequences to be amplified for example lies between 5 and 8, then frequency distribution curves can be obtained which permit a good distinction of a copy number of the predetermined sequence per cell of 0, 1 or larger than equal to 2, whereas the adaptation to 8 to 12 sequences which are not homologous to one another enables a reliable statement on whether the predetermined sequence, for example a specific chromosome or specific gene is present per cell in a copy number of 0, 1, 2 or greater than or equal to 3.

In a further development of the concept of the invention it is proposed to select the number of the copies of the predetermined sequence to be determined in the biological sample to be between 0 and 100, preferable between 0 and 25, particularly preferred between 0 and 10 and quite especially preferred between 0 and 5.

In accordance with a further preferred embodiment of the present invention, only one PCR is carried out in step b) of the method of the invention, with a number of primer pairs which are adapted to amplify the at least two sequences not homologous to one another being used in the PCR corresponding to the number of the at least two sequences not homologous to one another. An advantage of this embodiment lies in the fact that only one PCR is required both for the recording of the frequency distribution(s) and also for carrying out the amplification reaction in step b), so that the method can be carried out rapidly and without a large pipetting cost and complexity. An example for a suitable way of carrying out the method is a multiplex PCR; however, any other amplification reaction can also be used in which the at least two sequences to be amplified which are not homologous to one another are simultaneously amplified in one reaction.

In accordance with a further preferred embodiment of the present invention an individual PCR is carried out for each of the at least sequences which are not homologous to one another and which are to be amplified, so that in each PCR only one primer pair is used. For the realization of this embodiment a number of aliquots of a biological sample is made available in step a) of the method of the invention corresponding to the number of the at least two sequences which are not homologous to one another, with each aliquot containing the same quantity of biological material before the aliquots are used in the individual PCRs. An advantage of this way of carrying out the method lies in the fact that the individual amplifications cannot mutually influence each other. In this embodiment it can be necessary, in particular with only a small quantity of biological sample, for example if only one cell is available, to amplify the biological sample with a non-specific PCR prior to carrying the method of the invention and to divide the so obtained reaction product into the required number of aliquots. Naturally, in this way of carrying out the method, the reference samples must be pre-amplified in the same way for the recording of the frequency distribution and portioned into aliquots.

Finally, provision is made in accordance with a further preferred embodiment of the present invention to amplify a part of the at least two sequences which are not homologous to one another in one PCR and the other part of the at least two sequences which are not homologous to one another and which are to be amplified in respective PCRs separate therefrom, with in each case only one primer pair being used in these PCRs. Consequently, this way of carrying out the method is a mixed form of the previously named method forms.

A particular advantage of the method in accordance with the invention lies in the fact that in step c), in the determination of the number of different PCR products per amplification batch at least two pieces of information are taken into account per amplification batch, namely on the one hand the presence or absence of the corresponding PCR product and on the other hand the information concerning a second parameter which distinguishes the individual PCR products from one another, for example the length of a sequence of the PCR products, which is why a surprising sharp frequency distribution is obtained in comparison to a corresponding method in which only the presence or absence of the individually obtained PCR products is taken into account.

For the determination of the absence or presence of the at least two sequences which are not homologous to one another and which are to be amplified any suitable method available to the person skilled in the art for this purpose can be used, with gel electrophoresis, customary hybridization techniques, for example hybridization methods on a DNA array, a bead system and also other optical electrical or electrochemical measurements are named purely by way of example. In this connection it can be expedient, in dependence on the detection method which is used, to define threshold values above which the presence of a PCR product and below which the absence of a PCR product is assumed. The nature of a second parameter which individualizes the individual PCR products from one another depends essentially on the type of the at least two sequences which are not homologous to one another which have to be amplified. If, for example, the PCR primers are so selected in the at least one amplification reaction that STR sections and/or VNTR sections are amplified as sequences which are not homologous to one another, the length of the individual PCR products is preferably selected as the second parameter or as the distinguishing feature of the individual PCR products, so that the determination of the number of the different amplification products obtained in accordance with step c) includes the examination for the presence or absence of PCR products and also the determination of the length of the individual PCR products, with the number of the different amplification products that are received, corresponding to the number of the amplification products of different length that are received. A suitable method for this is for example capillary electrophoresis.

If, in contrast, PCR primers are used in the at least one amplification reaction which are adapted to amplify at least two SNP sequences which are not homologous to one another, then the second distinguishing feature, or the second parameter, is preferably the determination of the differing sequence, which is normally restricted in SNP sections to a nucleotide. For this purpose, all methods known to the person skilled in the art for this purpose can be used, with DNA sequencing or known hybridization methods being named simply by way of example. In this embodiment the number of the different amplification products that are obtained thus conesponds to the number of the obtained amplification products with differing sequence.

In accordance with a further preferred embodiment the present invention relates to a method for the quantitative determination of the copy number of a predetermined nucleic acid sequence and sequences homologous thereto in a cell which includes the following steps:
a) making available a biological sample, with the biological sample including between 1 and 100 cells and/or 1 pg and 100 pg chromosomal DNA,
b) carrying out at least one PCR, with the at least one PCR being adapted to amplify at least two sequences which are not homologous to one another and which are included by the predetermined sequence, which are selected from the group comprising STR sections, VNTR sections, SNP sections and arbitrary combinations hereof,
c) determination of the number of the different amplification products that are obtained and also
d) comparison of the number of different amplification products obtained with at least one frequency distribution which was or is obtained by separate and in each case multiple carrying out of the same at least one PCR and under the same reaction conditions as used in step b), with the same quantity of starting material having been used in the PCRs as named in step a), with at least two different reference samples, with the at least two different reference samples respectively having a known copy number of the predetermined sequences which are different from one another as well as subsequent determination of the number of different amplification products received per reference sample.

Since the method of the invention is a statistical method it is advantageous to set the starting quantity and the PCR conditions, in particular with respect to the temperature control, the number of cycles and the binding affinity of the primer in such a way that the individual PCR reactions effected in parallel take place with a relative frequency for a positive result of greater than 0 but smaller than 1. In this way it is ensured that a statistical evaluation is achieved with the highest possible security and reliability of the result that is obtained with a minimum of experimental effort. Thus it is preferred to set the binding affinity of the individual PCR primers to their primer binding sites and also the other parameters of the PCR, in particular the number of cycles and the temperature control, in such a way that the relative frequency for a positive amplification reaction of the at least one amplification reaction for each of the at least two sequences which are not homologous to one another and which are to be amplified amounts to between 0.2 and less than 1 and particularly preferably between 0.4 and 0.6 and especially preferred to approximately 0.5.

In particular, when the frequency distribution was recorded prior to the steps a) to c) of the method of the invention, it has proved expedient to carry out in parallel to the at least one amplification reaction in accordance with step b) an amplification reaction under the same conditions with a control sample, with the control sample preferably leading to a known number of different amplification products. In this way it is possible to determine in a simple manner whether the at least one amplification reaction in accordance with step b) has taken place in an orderly manner, or whether it has possibly not taken place at all or only inadequately due to a defect at the thermo-cycler.

As a further development of the concept of the invention it is proposed to use a pole body as a biological sample, preferably a pole body after the first meiosis. Since the method of the invention is in particular suitable for the quantitative determination of a copy number of a predetermined sequence and of sequences homologous thereto per cell, in particular for the quantitative determination of the copy number of alleles per cell of 0, 1 or larger than or equal to 2 or of 0, 1, 2 or larger than or equal to 3, it is excellently suited to allow conclusions through a pole body analysis regarding the genome of the corresponding ovum from which the pole body was taken. This is therefore of great significance in prenatal diagnostics because then any chromosome aberrations can already be recognized prior to in vitro fertilisation whereas with other known methods such as for example the amniocentesis corresponding faulty distributions of chromosomes can only first be determined at a much later time.

During the ripening of the ovum the chromosome set of the initially diploid ovum is reduced to a haploid chromosome set. Whereas the homologous chromosomes are separated during the first meiosis, with a haploid chromosome set remaining in the ovum and the other being separated out in the form of the pole body, in the second meiosis the separation of the individual chromatides of the chromosomes remaining in the ovum takes place, with a set of chromatides in the form of the second pole body being expelled from the ovum whereas the other set of the chromatides remains in the ovum. The two pole bodies transferred during the two meioses from the ovum into the perivitellinen gap of the ovum thus correspond in their genetic make-up to a cell but have however only a minimum proportion of cytoplasm. Whereas the first pole body arises during the ovulation the second pole body is first expelled three to four hours after the penetration of the sperm into the ovum. Since pole bodies do not have any function at all and are in any event resorbed in the early development of the embryo the removal of a pole body from the ovum is possible, on the one hand without damaging the ovum and without the danger of a negative influence of the further development and, on the other hand, is permissible for the pole body after the first meiosis in accordance with the German law relating to the protection of the embryo. Thus the investigation of the pole body offers as a whole the possibility of diagnosing possible faulty distribution of chromosomes in the ovum at a very early stage, namely prior to fertilisation of the ovum.

With the method of the invention it is possible to rapidly simply and reliably diagnose faulty distributions of chromosomes in an ovum by the invesligation of the pole body taken from it after the first meiosis. For this a single pole body can for example be subjected to a PCR, with the PCR being designed as a multiplex PCR in which for example eight different primer pairs are used which are adapted to amplify eight STR sequences which are not homologous to one another which are contained on the chromosome to be investigated, for example the chromosome 21. As an alternative to this it is naturally also possible to amplify the eight STR sequences which are not homologous to one another separately in each case in eight different PCRs. The following possibilities exist with regard to the genetic outfit of the ovum with respect to the chromosome 21:
1. the cells contains three like alleles (mono-allelic trisomy),
2. the cell contains two like alleles and an allele different there from (bi-allelic trisomy),
3. the cell contains three different alleles (tri-allelic trisomy),
4. the cell contains two like alleles (mono-allelic disomy [healthy homozygote cell]),
5. the cell contains two different alleles (bi-allelic disomy [healthy heterocygote cell]) or
6. the cell contains no allele of chromosome 21.

In accordance with the invention a frequency distribution is first prepared with at least two different reference samples with, for example, a PCR being carried out in each case 100 times for each reference sample with the primer pairs for the amplification of, for example, 8 STR sequences which are not homologous to one another. As reference samples six different pole bodies respectively having one of the above named genetic make-ups can, for example, be used. If it is only desired to distinguish between a mono-allelic disomy and a bi-allelic disomy, then it is naturally sufficient when the two corresponding reference samples are used to generate the frequency distribution. Subsequently, or in parallel to the recording of the frequency distribution curves, a pole body to be investigated can then be subjected to the same multiplex PCR and the copy number determined by comparison of the received number of different PCR products with the frequency distribution.

As already indicated, the resolution of the frequency distribution can be set to the desired value by adaptation of the method conditions, for example the number of the sequences which are not homologous to one another which are to be amplified and the number of the PCR cycles, so that overlaps of the frequency distribution curves can be avoided in the range of interest.

This should be explained with the example of the following conceptual experiment. One proceeds in the same way as explained above with reference to Table 1, with the exception that the PCR carried out with the reference samples and with the biological sample to be investigated being carried out in each case with 12 instead of 8 suitable primer pairs for an amplification of STR sequences which are not homologous to one another. The following frequency distribution is for example obtained:

TABLE 3
(Frequency distribution with 12 STR systems and high cycle number)

| Copy number | Number of different PCR products obtained | | | | | | | | | | | | |
|---|---|---|---|---|---|---|---|---|---|---|---|---|---|
| of the RS | 0 | 1 | 2 | 3 | 4 | 5 | 6 | 7 | 8 | 9 | 10 | 11 | 12 |
| N = 0 | 95 | 5 | 0 | 0 | 0 | 0 | 0 | 0 | 0 | 0 | 0 | 0 | 0 |
| N = 1 | 0 | 0 | 3 | 20 | 44 | 30 | 3 | 0 | 0 | 0 | 0 | 0 | 0 |
| N = 2 | 0 | 0 | 0 | 0 | 0 | 0 | 0 | 3 | 27 | 45 | 15 | 7 | 3 |

n = number of copies of a predetermined sequence in a reference sample
RS = reference sample

As can be deduced from Table 3, the number of sequence copies can be unambiguously determined with this frequency distribution for each result obtained for the sample to be investigated. By increasing the number of non-homologous sequences which are to be amplified from 8 to 12, a spreading of the individual frequency distribution curves is thus achieved while avoiding a partial overlap of the individual frequency distribution curves, such as is the case in the conceptual experiment indicated with reference to Table 1 for the result of one of four different PCR products.

A further important parameter which influences the resolution of the frequency distribution is the cycle number that is used for the at least one PCR. If, for example, in the conceptual test explained above with reference to Table 3, the cycle number of the PCR is reduced from 30 to 25 with otherwise the same reference samples and PCR conditions then, for example, the frequency distribution reproduced in Table 4 is obtained:

TABLE 4
(Frequency distribution with 12 STR systems and a low number of cycles)

| Copy number | Number of different PCR products obtained | | | | | | | | | | | | |
|---|---|---|---|---|---|---|---|---|---|---|---|---|---|
| of the RS | 0 | 1 | 2 | 3 | 4 | 5 | 6 | 7 | 8 | 9 | 10 | 11 | 12 |
| n = 0 | 98 | 2 | 0 | 0 | 0 | 0 | 0 | 0 | 0 | 0 | 0 | 0 | 0 |
| n = 1 | 0 | 1 | 14 | 22 | 40 | 20 | 3 | 0 | 0 | 0 | 0 | 0 | 0 |
| n = 2 | 0 | 0 | 0 | 0 | 0 | 0 | 3 | 10 | 30 | 32 | 15 | 7 | 3 |

n = number of copies of a predetermined sequence in a reference sample
RS = reference sample

In comparison to the frequency distribution reproduced in Table 3 the individual frequency distribution curves in Table 4 lie closer to one another and intersect at least partly. This conceptual experiment thus allows a clear statement concerning the number of copies only in those cases in which 0 PCR products, 2 to 5 different PCR products or 7 or more PCR products are obtained with the cell to be investigated, with the copy number of chromosome 21 in the biological sample being—with the required reliability—0 for 0 PCR products, 1 for 2 to 5 PCR products, and 2 for 7 and more different PCR products. In contrast, no clear pronouncement as to how many copies of chromosomes 21 the sample actually contains is possible when 1 or 6 different PCR products are obtained for the samples to be investigated.

As a person skilled in the art will recognize, the width of the frequency distribution curves and the spacing of the different frequency distribution curves to one another can be set almost as arbitrarily by the setting of the PCR conditions and by the determination of the number of the at least two sequences which are not homologous to one another and which have to be amplified.

A further subject of the present invention is a kit for the quantitative determination of the number of a predetermined sequence and optionally of sequences homologous thereto in a biological sample which is suitable for carrying out the method of the invention. In accordance with the invention this kit includes:
a) at least two primer pairs which are adapted to amplify in at least one PCR at least two sequences which are not homologous to one another which are included by the predetermined sequence,
b) optionally a PCR buffer,
c) a protocol for the carrying out of the at least one PCR and
d) at least one frequency distribution which was obtained by separate and in each case multiple carrying out of the same at least one amplification reaction prescribed for the biological sample to be investigated in the protocol c) and under the same reaction conditions, wherein, in the amplification reaction, the same quantity of starting material as prescribed in the protocol c) was used, with at least two different reference samples, with the at least two different reference samples respectively having a known copy number of the predetermined sequence different from one another and also with subsequent determination of the number of different amplification products obtained per reference sample.

In accordance with a preferred embodiment of the kit in accordance with the present invention the at least two primer pairs are adapted to amplify in the at least one PCR at least two sequences which are not homologous to one another from the non-coded DNA range. Good results are obtained in particular when the two non-homologous sequences are polymorphous to a high degree. Particularly preferred is when the at least two primer pairs are adapted to amplify selected sequences not homologous to one another from the group consisting of STR sequences, VNTR sequences, SNP sequences and any desired combinations hereof.

In a further development of the concept underlying the invention it is proposed that the at least primer pairs and/or the protocol are adapted in such a way that in the PCR 2 to 100, particularly preferably 2 to 20, quite especially preferred 3 to 15 and most preferred 5 to 12 sequences which are not homologous to one another are amplified.

For the already explained reasons it is, moreover, preferred to adapt the at least two primer pairs and/or the protocol so that the relative frequency of the at least one PCR for each of the at least two sequences which are not homologous to one another amounts to between 0.2 and less than 1, particularly preferred to between 0.4 and 0.6 and quite especially preferred to approximately 0.5.

Moreover, it has proved to be advantageous to adapt the protocol of the PCR and/or the at least one frequency distribution to determine whether the biological sample has the predetermined sequence in a copy number per cell of 0, 1 or at least 2 or in a copy number per cell of 0, 1, 2 or at least 3.

Furthermore the present invention relates to an apparatus which is in particular suitable for carrying out the method of the invention comprising:
a) a fixed support, in particular a glass support,
b) at least two primer pairs which are immobilized on the support and which are adapted to amplify in at least one PCR at least two sequences which are not homologous to one another which are included by the predetermined sequence and also
c) a stored frequency distribution, e.g. an electronically stored frequency distribution, which was obtained by separate and in each case multiple carrying out of at least one amplification reaction with at least two different reference samples, with the at least two different reference samples respectively having a known copy number of the predetermined sequence which are different from one another and also subsequent determination of the number of different amplification products obtained per reference sample or
d) at least two reference samples immobilized spatially separate from one another on the support, with the at least two different reference samples respectively having a known copy number of the predetermined sequence different from one another.

The primer pairs and/or the reference samples are preferably immobilized on the carrier via non-chemical bonds.

In the following the invention will be explained with reference to an example which explains the concept of the invention but does not restrict it.

### EXAMPLE

### Preparation of a Frequency Distribution

### a) Carrying Out of a PCR Reaction

Individual lymphocytes of an individual were deposited under microscopic control as reference samples on the individual amplification anchors of an AmpliGrid glass support, a glass strip commercially sold by the company Advalytix AG for carrying out parallel automated PCR reactions, with an amplification anchor for example being a part area which has been pretreated in such a way that a liquid preferentially stays thereon. In this connection 1 to 6 lymphocytes are deposited per amplification anchor with negative checks consisting of system fluid without the lymphocytes being additionally placed on some anchors. In this connection multiple determinations were carried out in each case, with 21 negative controls without lymphocytes, 27 samples having one lymphocyte each, 42 samples with two lymphocytes each, 35 samples with three lymphocytes each, 31 samples with four lymphocytes each, 17 samples with five lymphocytes each and 6 samples with 2 lymphocytes each, i.e. a total of 175 samples were distributed on the glass support.

Thereafter a 1 μl aliquot of the following composition was pipetted onto the individual amplification anchors provided with the samples from a PCR master mix.

| Component | Quantity in μl |
|---|---|
| 10 x PCR buffer (QIAGEN) | 0.1 |
| Hot Star Taq (QIAGEN) | 0.096 |
| Mixture of primer pairs | 0.1 |
| (PowerPlex 16 PCR-Kit [Promega]) | |
| dNTP mixture (each 2.5 mM) | 0.1 |
| Water (twice distilled) | 0.604 |
| Total quantity | 1 |

Thereafter the individual PCR drops were coated with 5.2 μm mineral oil in each case (Covering Solution, company Advalytix AG). For this the mineral oil was first so pipetted that it hung in the form of a droplet on the tip of the pipette before the PCR drop was contacted with this droplet until the PCR drop was uniformly covered with the mineral oil.

Following this a PCR with the following temperature profile was carried out with the individual samples:

| | | |
|---|---|---|
| Temperature | Time per cycle | Cycle number |

| | | | |
|---|---|---|---|
| 95° C. | 15 | min. | |
| 96° C. | 1 | min. | |
| 94° C. | 30 | sec. | 10 |
| 60° C.* | 30 | sec. | |
| 70° C.** | 45 | sec. | |
| 90° C. | 30 | sec. | 20 |
| 60° C.* | 30 | sec. | |
| 70° C.** | 45 | sec. | |
| 60° C. | 30 | min. | |
| 20° C. | 5 | min. | |

| | |
|---|---|
| 8° C. | ∞ |

*0.5° C./sec. ± 0.0° C./sec.
**0.3° C./sec. ± 0.0° C./sec.

After the amplification the total volume of the individual reactions was mixed in each case with 4 μl water (twice distilled). In this the water mixed with the aqueous phase of the sample. The total reaction volume including mineral oil was then transferred into various containers of a microtiter plate.

### b) Determination of the Number of Different PCR Products Obtained

The individual reaction volumina of the microtiter plate were respectively briefly denatured with in each case 19 μl formamide+1 μl ILS 600 (internal fluorescence standard, company Applied Biosystems) and separated electrophoretically under standard conditions in a capillary sequencer ABI 3100 (company Applied Biosystems). The results were stored in the form of Genotyper files, with the recognition of relevant signals and their association to the standard taking place automatically by the Genotyper software (company Applied Biosystems). In known manner the fluorescence signals are always measured against a background of fluorescence and, as in every measurement, the signal-to-noise ratio is decisive. This is not set out by the software. As the lower limit (threshold value) of a relevant signal 500 relative luminescent units were specified in this experiment.

The automatically recognizes alleles per amplification were counted for all systems and the number of positive reactions set in relation to the cell number.

In this connection the following result was obtained:

| Lymphocyte cells/anchor | N | Mean | Standard deviation |
|---|---|---|---|
| 0 | 21 | 0.048 | 0.22 |
| 1 | 27 | 10.04 | 6.72 |
| 2 | 42 | 14.00 | 7.07 |
| 3 | 35 | 17.86 | 5.87 |
| 4 | 31 | 19.48 | 5.63 |
| 5 | 17 | 23.24 | 3.96 |
| 6 | 2 | 25.50 | 0.71 |
| Total | 175 | 14.49 | 8.72 |

In the table:
Lymphocyte cells/anchor: Signifies the number of the deposited cells per amplification anchor. In the case of diploid lymphocytes that is precisely 2 copies of a homologous sequence per cell. These two copies are available for the PCR as a starting template and can both be amplified. If the two copies are sequence-identical then one determines precisely one peak during the capillary electrophoresis. If the two copies differ in length (heterocygote case) then two different lengths of a homologous sequence can be amplified. Thus two peaks arise.
The here analyzed groups 0, 1, 2, 3, 4, 5, 6 are distinguished in the number of starting copies i.e. by in each case 2 copies (1 cell: 2 copies, 2 cells: 4 copies, etc.).
N: Signifies the number of the individual reactions which were started with a defined cell number. Since the cells are randomly deposited there are different random sample sizes. The case “6 cells” was only presented twice, the case “5 cells” 17 times etc. The statistical analysis takes this into account.
Mean: Signifies the average value of the number of positive signals (peaks, which were found automatically with software support), i.e. the average value of the number of different PCR products.
Standard deviation: Signifies the standard deviation from the above-named average value as a measure for the scatter around the average value. The subsequently described variance analysis (ANOVA) utilizes these three parameters in order to calculate the statistically significant distinctions and to make a pronouncement as to whether the average values are actually different. Statistically spoken one wishes to check the hypothesis whether for example for the random samples “2 cells presented” and “4 cells presented” are different basic totalities.

For the case “0 copies presented” the case of a single peak was detected once in 21 experiments. This is a false positive result which came about by
a. a contamination with human DNA; however the PowerPlex Kit combines up to 16 different sequence sections in one reaction. A contamination would thus have to have to taken place with a single sequence (physically for example one chromosome) which is very improbable or in that
b. the capillary electrophoresis incorrectly showed a signal; this can be a voltage pulse during electrophoresis or a contamination in the gel by a fluorescence particle of unknown origin which is interpreted as a peak.

In any event a positive value thus results for the case “0 copies presented” and a positive standard deviation. Without a false positive signal the average value and the standard deviation would be 0.

The last line “total” sums the above-named values.

### c) Variance Analysis Over all Data

A variance analysis (ANOVA) was carried out with the above-named data. The variance analysis relates to a comparison of the variances (or standard deviations) within a group (within groups) with the variances between the groups (between groups). In this connection the following result was obtained:

| | Sum of | | Mean | | |
|---|---|---|---|---|---|
| | Squares | Difference | Square | F | Significance |
| Between | 7638.212 | 6 | 1273.035 | 38.194 | 0.0000 |
| Groups | | | | | |
| Within | 5599.502 | 168 | 33.330 | | |
| Groups | | | | | |
| Total | 13237.714 | 174 | | | |

In this respect:
Sum of Squares is the sums of the squares of the deviation
Df is the number of degrees freedom and
Mean Square is the average sums of the square deviations
The F-value finally sets deviations within the groups and between the groups in relationship to one another (1273.035/33.33 = 38.194). Whether the value for a specific number of degrees of freedom shows a significant distinction between the groups can be looked up in a table (for example J. Bortz: Statistik für Sozialwissenschaftler (statistics for social scientists), Springer Press). In this case the distinction is highly significant (P = 0.000).

This result shows that there are highly significant differences between the different groups of different cell numbers.

### d) Scheffe Test

In order to make a pronouncement as to between which groups the difference really exists, a Scheffe test was carried out with the results that are obtained. The result of the test in accordance with Scheffe recites the groups between which significant differences are present with pair-wise comparison. In this respect the average differences between two groups (mean difference I-J) and also their standard error (Std. Error) are shown. A significance limit of 0.05 was selected.

The following results were obtained.

| | | Mean | | | Groups |
|---|---|---|---|---|---|
| (I) | (J) | Difference | Std. | Signif- | distin- |
| NOCELLS | NOCELLS | (I − J) | Error | icance | guishable? |
| 0 | 1 | −9.99 | 1.68 | .000 | Yes |
| | 2 | −13.95 | 1.54 | .000 | Yes |
| | 3 | −17.81 | 1.59 | .000 | Yes |
| | 4 | −19.44 | 1.63 | .000 | Yes |
| | 5 | −23.19 | 1.88 | .000 | Yes |
| | 6 | −25.45 | 4.27 | .000 | Yes |
| 1 | 0 | 9.99 | 1.68 | .000 | Yes |
| | 2 | −3.96 | 1.42 | .264 | No |
| | 3 | −7.82 | 1.48 | .000 | Yes |
| | 4 | −9.45 | 1.52 | .000 | Yes |
| | 5 | −13.20 | 1.79 | .000 | Yes |
| | 6 | −15.46 | 4.23 | .043 | No |
| 2 | 0 | 13.95 | 1.54 | .000 | Yes |
| | 1 | 3.96 | 1.42 | .264 | No |
| | 3 | −3.86 | 1.32 | .210 | No |
| | 4 | −5.48 | 1.37 | .016 | Yes |
| | 5 | −9.24 | 1.66 | .000 | Yes |
| | 6 | −11.50 | 4.18 | .277 | No |
| 3 | 0 | 17.81 | 1.59 | .000 | Yes |
| | 1 | 7.82 | 1.48 | .000 | Yes |
| | 2 | 3.86 | 1.32 | .210 | No |
| | 4 | −1.63 | 1.42 | .971 | No |
| | 5 | −5.38 | 1.71 | .135 | No |
| | 6 | −7.64 | 4.20 | .767 | No |
| 4 | 0 | 19.44 | 1.63 | .000 | Yes |
| | 1 | 9.45 | 1.52 | .000 | Yes |
| | 2 | 5.48 | 1.37 | .016 | Yes |
| | 3 | 1.63 | 1.42 | .971 | No |
| | 5 | −3.75 | 1.74 | .592 | No |
| | 6 | −6.02 | 4.21 | .915 | No |
| 5 | 0 | 23.19 | 1.88 | .000 | Yes |
| | 1 | 13.2 | 1.79 | .000 | Yes |
| | 2 | 9.24 | 1.66 | .000 | Yes |
| | 3 | 5.38 | 1.71 | .135 | No |
| | 4 | 3.75 | 1.74 | .592 | No |
| | 6 | −2.26 | 4.32 | 1.000 | No |
| 6 | 0 | 25.45 | 4.27 | .000 | Yes |
| | 1 | 15.46 | 4.23 | .043 | Yes |
| | 2 | 11.50 | 4.18 | .277 | No |
| | 3 | 7.64 | 4.20 | .767 | No |
| | 4 | 6.02 | 4.21 | .915 | No |
| | 5 | 2.26 | 4.32 | 1.000 | No |

* The mean difference is significant at the .05 level.

The table is to be interpreted as follows:

Those groups between which the “significance” amounts to less than 0.05 are significantly different from one another whereas in the other groups no distinction is possible at the selected significance level.

For example the group “0 cells” is significantly different from all others (first block). A qualitative decision is thus possible (0 copies in comparison to all other cases).

The group “1 cell” is distinguished from the groups “0 cells”, “3 cells”, “4 cells”, “5 cells” and “6 cells”. A distinction 1 cell/2 cells is in contrast not possible because the “significance” between these groups amounts to 0.264.

By changing the PCR conditions, in particular the cycle number, the individual groups can be distinguished from one another in accordance with the requirements in that they are significantly different from one another.

1-39. (canceled)

40. A method for the quantitative determination of the number of a predetermined sequence and optionally of sequences homologous to the predetermined sequence in a biological sample, in particular for the determination of the absolute number of copies of alleles per cell, the method including the steps of:
a) making available a defined quantity of a biological sample, which contains less than one of 100 pg DNA and 100 cells,
b) carrying out at least one amplification reaction, with the at least one amplification reaction being adapted to amplify at least two sequences which are not homologous to one another and which are included in the predetermined sequence,
c) determination of the number of the different amplification products that are obtained and also
d) comparison of the number of the different amplification products obtained with at least one frequency distribution which was or is obtained by separate and in each case multiple carrying out of the same at least one amplification reaction and under the same reaction conditions as used in step b), with the same quantity of starting material having been used or being used in the amplification reaction as in step a) with at least two different reference samples, with the at least two different reference samples respectively having a known copy number of the predetermined sequences which are different from one another as well as subsequent determination of the number of different amplification products which was or is received per reference sample, wherein the at least one amplification reaction is adapted to amplify at least one of:
i) at least two sequences which are not homologous to one another and which are selected from the group consisting of STR-sequences, VNTRsequences, SNP-sequences and arbitrary combinations thereof and
ii) at least two non-homologous sequences which are only present once per allele in the genome of the donor.

41. A method in accordance with claim 40, wherein the predetermined sequence is a nucleic acid sequence, preferably a chromosome, a gene or a gene section.

42. A method in accordance with claim 40, wherein the at least one amplification reaction is a PCR reaction.

43. A method in accordance with claim 40, wherein the biological sample made available in step a) includes one of less than 50 pg DNA, less than 10 pg DNA and less than 5 pg DNA.

44. A method in accordance with claim 40, wherein the biological sample used in step a) includes one of less than 10 cells, less than 5 cells and 1 cell.

45. A method in accordance with claim 40, wherein the frequency distribution consists of frequency distribution curves obtained with at least two reference samples with defined copy numbers different from one another obtained at the predetermined sequence, with each of the frequency distribution curves setting forth the probability for receiving each number lying between zero and a theoretically possible maximum number of different PCR products for a defined copy number.

46. A method in accordance with claim 40, wherein the frequency distribution consists of the recitation of average values of the number of different PCR products obtained with the individual reference samples with defined copy numbers different from one another of the predetermined sequence during the multiple determination.

47. A method in accordance with claim 46 wherein said recitation includes the standard deviation.

48. A method in accordance with claim 40, wherein a number of reference samples is used for the determination of the frequency distribution, said number being in the range from 3 to 20, said reference samples being used with known respectively differing copy numbers of the predetermined sequence.

49. A method in accordance with claim 40, wherein the at least one amplification reaction used to establish the frequency distribution used in step d) is carried out a number of times selected to lie in the range from 2 to 1000 for each reference sample.

50. A method in accordance with claim 40, wherein the establishment of the frequency distribution takes place in parallel to the carrying out of the steps a) and c) or has already been carried out prior to step a).

51. A method in accordance with claim 40, wherein the at least one amplification reaction is adapted to amplify a number of sequences which are not homologous to one another lying in the range from 2 to 100.

52. A method in accordance with claim 40, wherein the number of the copies to be determined of the predetermined sequence in the biological sample lies in the range from 0 to 100.

53. A method in accordance with claim 40, wherein a PCR is carried out in step b) in which a number of primer pairs is used corresponding to the number of the at least two sequences which are not homologous to one another, with the primer pairs being adapted to amplify the at least two sequences which are not homologous to one another.

54. A method in accordance with claim 40, wherein, in step a), a number of aliquots of a biological sample corresponding to the number of the at least two sequences which are not homologous to one another is made available, with each aliquot containing the same quantity of biological material and wherein, in step b), a PCR is carried out with each of the aliquots in which in each case one primer pair is used, with the primer pairs used in the various PCRs being adapted to amplify the at least two sequences which are not homologous to one another.

55. A method in accordance with claim 40, wherein, in step a), at least two aliquots of a biological sample are made available, but a number of aliquots of the biological sample which is smaller than the number corresponding to the number of the at least two sequences which are not homologous to one another, with each aliquot containing the same quantity of biological material and wherein, in step b), a PCR is conducted with each of the aliquots in which in each case at least one primer pair is used but a smaller number of primer pairs than that which corresponds to the number of the at least two sequences which are not homologous to one another, with the primer pairs used in the different PCR's being adapted to amplify the at least two sequences which are not homologous to one another.

56. A method in accordance with claim 40, wherein the biological sample is amplified with a non-specific PCR prior to carrying out the method step a).

57. A method in accordance with claim 56, wherein the reaction product obtained by the non-specific PCR is subdivided into the required number of aliquots.

58. A method in accordance with claim 40, wherein the presence or absence of amplification products is determined by means of at least one of the following: gel electrophoresis, a hybridization technique on a DNA array, a hybridization technique on a bead system, an optical measurement, an electrical measurement and an electrochemical measurement.

59. A method in accordance with claim 40, wherein, for the determination of the number of different amplification products that are obtained after the amplification reaction, the presence or absence of the at least two sequences which are not homologous to one another is determined and also a second parameter is determined from the obtained amplification products, said second parameter being at least one of physically and chemically measurable.

60. A method in accordance with claim 59, wherein the at least two sequences which are not homologous to one another are selected from at least one of STR sections and VNTR sections and a length of the obtained amplification products is determined as the second parameter, with the number of the different amplification products that are obtained corresponding to the number of the obtained amplification products with differing length.

61. A method in accordance with claim 60, wherein the length of the amplification products is determined by capillary electrophoresis.

62. A method in accordance with claim 61, wherein the at least two sequences which are not homologous to one another are SNP sections and wherein the sequence of the obtained amplification products is determined as the second parameter, with the number of the different amplification products that are obtained corresponding to the number of the obtained amplification products with different sequence.

63. A method in accordance with claim 62, wherein the sequence of the amplification products is determined by one of DNA sequencing and a hybridization method.

64. A method for the quantitative determination of the number of a predetermined nucleic acid sequence and of sequences homologous thereto in a cell, the method including the steps of:
a) making available a biological sample, with the biological sample including at least one of between 1 and 100 cells and between 1 pg and 100 pg chromosomal DNA,
b) carrying out at least one PCR, with the at least one PCR being adapted to amplify at least two sequences which are not homologous to one another, which are included by the predetermined sequence and which are selected from the group comprising STR sections, VNTR sections, SNP sections and arbitrary combinations hereof,
c) determination of the number of the different amplification products that are obtained and also,
d) comparison of the number of different amplification products obtained with at least one frequency distribution which was or is obtained by separate and in each case multiple carrying out of the same at least one PCR and under the same reaction conditions as used in step b), with the same quantity of starting material having been used in the PCRs as named in step a), with at least two different reference samples, with the at least two different reference samples respectively having a known copy number of the predetermined sequence which are different from one another as well as subsequent determination of the number of different amplification products received per reference sample, wherein the at least one PCR is adapted to amplify at least one of:
i) at least two sequences which are not homologous to one another and which are selected from the group consisting of STR-sequences, VNTRsequences, SNP-sequences and arbitrary combinations thereof and
ii) at least two non-homologous sequences which are only present once per allele in the genome of the donor.

65. A method in accordance with claim 40, wherein an amplification reaction is carried out with a control sample under the same conditions in parallel to the at least one amplification reaction in accordance with step b).

66. A method in accordance with claim 40, wherein a pole body is used as the biological sample.

67. A method in accordance with claim 66 wherein a pole body after the first meiosis is used.
